(19) United States
(12) Patent Application Publication
(10) Pub. No.: US 2026/0232426 A1
(43) Pub. Date: Aug. 13, 2026

(54) VASCULAR GRAFT ASSEMBLIES, VASCULAR GRAFT SYSTEMS, AND METHODS OF USE THEREOF

(71) Applicants: The Board of Trustees of the Leland Stanford Junior University, Stanford, CA (US); Yale University, New Haven, CT (US); Duke University, Durham, NC (US); Biograft, Inc., Stanford, CA (US)

(72) Inventors: Elbert Heng, Stanford, CA (US); Abhay Bangalore Ramachandra, Stanford, CA (US); Gloria Un Chyr, Stanford, CA (US); Taylor Klein, Durham, NC (US); Alison Marsden, Stanford, CA (US); Jack Boyd, Stanford, CA (US); Matthew Becker, Durham, NC (US); Jay Humphrey, Stanford, CA (US); Oluwatomisin Olurotimi Obafemi, Stanford, CA (US); Nathan M. Wilson, Santa Monica, CA (US); Maria Theresa Dulay, Stanford, CA (US); Joseph M. DeSimone, Stanford, CA (US)

(73) Assignee: The Board of Trustees of the Leland Stanford Junior University, Stanford, CA (US)

(21) Appl. No.: 19/469,898

(22) PCT Filed: Mar. 27, 2024
(86) PCT No.: PCT/US2024/021767

§ 371 Date: Sep. 26, 2025

Related U.S. Application Data
(60) Provisional application No. 63/492,427, filed on Mar. 27, 2023.

(51) Int. Cl. A61F 2/06

(52) U.S. Cl. CPC A61F 2/06; A61F 2210/0004; A61F 2210/0076; A61F 2230/0006; A61F 2230/0017; A61F 2230/0069; A61F 2240/002; A61F 2250/0067

(57) ABSTRACT

Reinforced external graft support assemblies and methods of manufacture and implantation are provided herein. In particular, graft support assemblies are composed of single or multilayer sheaths designed to provide mechanical support to vascular conduits during cardiovascular bypass surgery. Graft sheath systems may be fabricated using 3D printing techniques with biocompatible materials incorporate surgical design considerations such as mechanical strength, flexibility, porosity, and the ability to be tailored to specific anatomical structures. External graft sheaths may include one or more layers of various biomaterials to produce customized biomechanical properties including varied time courses of degradation. Furthermore, these customizable features may incorporate standardized designs or be tailored for individual patients depending on medical and surgical history, or other pertinent geometric attributes measured in pre-operative medical imaging. These methods protect vascular conduits, particularly venous grafts, from adverse remodeling and graft failure, thereby providing precision medicine solutions to cardiovascular bypass surgery.

### CROSS-REFERENCE TO RELATED APPLICATIONS

The present application claims priority to U.S. Provisional Application No. 63/492,427, filed Mar. 27, 2023, the full disclosure of which is incorporated herein by reference in its entirety for all purposes.

### STATEMENT OF GOVERNMENT SUPPORT

This invention was made with Government support under contracts HL159954, HL162397, and TR003142 awarded by the National Institutes of Health. The Government has certain rights in the invention.

### FIELD OF THE INVENTION

The present disclosure relates to grafts, in particular, external graft supports made from biocompatible materials that are designed for use in surgical procedures, including reinforced grafts fabricated using 3D printing techniques.

### BACKGROUND

Each year, approximately 400,000 coronary artery bypass grafting (CABG) surgeries, 55,000 arterio-venous fistula creation procedures, and 20,000 peripheral artery bypass surgeries are performed in the United States alone. Despite advances in medical technology and techniques for improving surgical outcomes of these procedures, graft failure remains a major problem costing the healthcare system billions of dollars per year due to repeat procedures leading to higher morbidity and mortality and lower quality of life for patients. Vein graft failure rates are as high as 50% at 10 years for CABG vein grafts and 25-55% at 5 years for infragenicular bypass grafts. Various approaches to prevent graft failure have been proposed including those disclosed in U.S. Patent Publications: 2012/0330437; 2014/0303715; U.S. Pat. Nos. 8,361,101; 9,265,632; 9,517,121; 9,579,224; yet currently none have satisfactorily resolved the considerable problem of graft failures. To improve upon current proposed solutions for reducing or avoiding graft failures, there is need for advanced materials that provide enhanced favorable mechanical properties such as flexibility, strength, and degradation and better biocompatibility; 3D printing techniques that allow precise control over dimensions; and surgical design considerations that facilitate ease of implementation during surgery and mitigate maladaptation postoperatively.

### BRIEF SUMMARY

The present disclosure relates to external sheaths configured to, among other things, provide support and reinforcement to a graft implanted via a coronary bypass procedure. The external sheaths disclosed herein may be biodegradable, bioabsorbable, customized, and/or adaptive to patient and/or surgical needs and/or may be configured to mitigate maladaptation and/or failure of vascular grafts and xenografts (autologous, or otherwise) after surgery. In some embodiments, the external sheaths described herein comprise novel materials with specialized properties to accommodate biocompatibility and 3D printing techniques. Additionally, or alternatively, the external sheaths and methods of use described herein may consider patient anatomy and/or surgical design considerations necessary for successful implementation. In some instances, the external sheaths and methods of use described herein may be advantageous for a variety of clinical applications, including, but not limited to, coronary bypass surgery, peripheral arterial disease treatment, and arteriovenous fistula creation. It is appreciated that the external sheaths, graft/external sheath assemblies and/or methods of use described herein may be applied to many different graft and/or implant applications in accordance with, for example, one or more of the concepts described herein.

Current surgical practices do little to aid in preventing maladaptation and/or adverse remodeling of a vascular graft after implantation, which plays a major role in graft failures largely due to sudden changes in biomechanical loading when transposed into arterial circulation during bypass surgery. The external sheaths disclosed herein may, for example, assist with supporting a graft so that it may experience a gradual change in mechanical loading may enable more favorable adaptation to arterial pressure, thus minimizing the risk of long-term graft failure.

The approaches described herein allow for production of external sheaths designed to mitigate maladaptation and failure of autologous vascular grafts and xenografts after surgery. In some embodiments, the external sheath design mitigates graft maladaptation by utilizing a custom-fit, non-blood contacting device (i.e., external sheath), made from biodegradable or bioresorbable materials. In some embodiments, the external sheath is comprised of biocompatible material that minimizes inflammatory response in the body and surgical conduit. In some embodiments, the external sheath material is biodegradable and maintains mechanical integrity for a set period of time (e.g. 1-6 weeks after surgery) before undergoing complete absorption by the body after external sheath is no longer beneficial to the surgical conduit (e.g. about 12 weeks after surgery or more). In other embodiments, the external sheath material may elute specific bioactive drugs to, for example, minimize immune and inflammatory responses, as well as promote favorable graft remodeling.

To successfully implement graft/external sheath assemblies in surgical practice, specific design considerations may be given to facilitate ease of intraoperative handling. For example, the external sheaths disclosed herein may utilize a single or multilayer, patient-specific, 3D-printed sheath composed of biocompatible, biodegradable, elastomeric, and/or drug eluting biomaterials to serve as an external support for prevention of vein graft failure. In some cases, the external sheath may be custom-designed and/or manufactured for the patient and/or may be customized and/or modified to fit the needs of a particular patient pre-operatively and/or during a surgical procedure. For specific surgeries and anatomic sites (i.e. CABG vs. peripheral bypass), the external sheaths disclosed herein may be manufactured with different degrees of curvature, diameter, and lengthwise and/or radial compressibility in order to, for example, prevent graft kinking, enable natural lay and also allow for successful completion of proximal and distal anastomoses with repair of anastomotic bleeding if needed. Depending on the surgical requirements, the external sheaths disclosed herein may incorporate different tessellating geometries (e.g., diamonds, auxetic hexagons, concatenated semicircles) in order to, for example, achieve intraoperative maneuverability during bypass surgery.

At times, to achieve efficient and customizable manufacturing and/or assembly of one or more of the external sheaths disclosed herein, novel biomaterials and/or formulations may be used to, for example, control mechanical stability and degradation behaviors thereof. In particular, poly(propylene fumarate) (PPF) is a bioresorbable aliphatic polyester capable of being produced in unique architectures to modulate its mechanical behavior that may be used to manufacture the external sheaths described herein so that they may be, for example, patient-specific, anatomy-specific, and/or surgery-specific. In some cases, thiol-ene chemistry techniques may be used to 3D print (using, for example, PPF) one or more of the external sheaths disclosed herein, which may allow for faster build times and/or more controllable properties than previously described techniques. At times, altering the alkene thiol ratio in PPF influences cross-linking density, mechanical properties, and desired sheath degradation times for gradually offloading different vascular conduits. In another aspect, adjusting resin viscosity optimizes 3D printing conditions for PPF material to form tubular support structures and reduce batch to batch variability.

### BRIEF DESCRIPTION OF DRAWINGS

FIGS. 1A-1H provide illustrations and renderings of exemplary external sheaths with various geometrical and/or strut designs, in accordance with some of the embodiments described herein.

FIG. 2A provides a side view of an exemplary external sheath, in accordance with some embodiments of the present invention.

FIG. 2B provides a top-perspective view of the exemplary external sheath of FIG. 2A, in accordance with some embodiments of the present invention.

FIGS. 3A-3F provide illustrations and renderings of exemplary external sheaths with different surface patterns, in accordance with some embodiments of the present invention.

FIGS. 4A-4C provide illustrations and renderings of exemplary multi-segmented external sheaths, wherein a pattern of the material of the comprising the respective external sheaths varies along its length, in accordance with some embodiments of the present invention.

FIGS. 5A-5E are illustrations of external sheaths that have varying cross sectional shapes, in accordance with some embodiments of the present invention.

FIGS. 6A and 6B are illustrations that show exemplary external sheaths that are specifically configured for directional compression/expansion, in accordance with some embodiments of the present invention.

FIG. 7 is a photograph of two exemplary external sheaths, in accordance with some embodiments of the present invention.

FIG. 8 is a photograph of an exemplary external sheath with a guide inserted therein along the external sheath's length, in accordance with some embodiments of the present invention.

FIG. 9 provides a flowchart of an exemplary process 900 for performing a CABG procedure using an external sheath, in accordance with some embodiments of the present invention.

FIG. 10A is a cross section view of an exemplary single layer graft/external sheath assembly, in accordance with some embodiments of the present invention.

FIG. 10B is a cross section view of an exemplary multi-layer graft/external sheath assembly, in accordance with some embodiments of the present invention.

FIG. 11 provides a photograph of an exemplary external sheath positioned over a graft in a manner consistent with execution of process 900, in accordance with some embodiments of the present invention.

### DETAILED DESCRIPTION

Coronary artery bypass grafting (CABG) surgery using venous grafts is performed in nearly half a million patients with multivessel or diffuse coronary artery disease each year in the United States. However, the venous grafts used in traditional CABG surgeries are problematic because the venous grafts often occlude and fail over time at a rate of 50% within 5-10 years after surgery, leading to repeat revascularization procedures, myocardial infarction, or death in 30% of patients within 5 years of the venous graft failure. It is well known that adverse remodeling of an implanted venous graft plays a major role in vein graft occlusion and failure, due in part to the sudden, drastic change in biomechanical loading when the venal tissue of the venous graft is transposed into the arterial circulation as a bypass graft, or conduit, and immediately faces higher levels of arterial blood pressure and higher shear stress from increased blood flow and pressure within the arterial circulation (as opposed to the venous circulation from which the graft originated). A gradual change in mechanical loading applied to the vein graft (as enabled by the external sheaths disclosed herein) may enable more favorable adaptation to arterial pressure and flow, thus improving venous graft performance and minimizing the risk of long-term venous graft failure. In some embodiments, the external sheaths and/or graft/external sheath assemblies and methods of use thereof disclosed herein may be deployed to, for example, support a venous graft used as a bypass conduit following implantation so that a gradual change in mechanical loading may be applied to the venous graft when positioned within an external sheath as disclosed herein, which may provide a favorable adaptive response after surgery in order to mitigate the above-noted problems and avoid or lessen the risk of graft failure. Surgical design considerations for vascular graft sheaths include distinct geometric patterning to, for example, allow external sheath curvature and flexibility, as well as radial and lengthwise elasticity and compressibility to accommodate proximal and distal anastomotic sites and lay of the graft. In some embodiments, the external sheath may be made from biocompatible, biodegradable and/or bioresorbable elastomeric biomaterials. Poly(propylene fumarate) (PPF) in particular is a bioresorbable aliphatic polyester we have applied to vascular sheath assemblies and can now be 3D printed using thiol-ene chemistry to achieve faster build times, customization of mechanical stability periods, and desired degradation behavior.

### I. Surgical and Mechanical Design Considerations

The approaches and design aspects described herein represent unique considerations for the construction of biodegradable and/or bioabsorbable external graft, or conduit, support devices (sometimes referred to herein as “external sheaths”) that may be tubes or sheaths configured to be positioned around an external surface of a graft and/or conduit inserted, for example, during coronary artery bypass grafting (CABG) surgery. The external sheaths disclosed herein may be configured to provide mechanical support to a venous, arterial, and/or synthetic graft used to bypass a damaged coronary artery while the graft remodels and/or adjusts to use as an arterial vessel, which may improve the graft's long-term durability and/or effectiveness. The external sheaths disclosed herein may be configured to incorporate one or more distinct mechanical (e.g., an elastic (Young's) modulus of 0.1-10 MPa) and/or chemical properties to, for example, provide desired stiffness/flexibility at various stages of graft remodeling, manipulation during surgical implantation, dissolution over time, and/or absorption into the body over time. Additionally, or alternatively, the external sheaths disclosed herein may be configured to enable ease of intraoperative handling for patient-specific, anatomy-specific, and/or surgery-specific applications and/or utilization.

In some embodiments, the external sheaths disclosed herein may comprise a biocompatible material selected and/or configured to minimize, or avoid, an undesirable inflammatory response of a patient's body to the graft and/or the external sheath. For example, the external sheaths disclosed herein may comprise a biodegradable material configured and/or selected to maintain a degree of stiffness, flexibility, and/or mechanical integrity for a set period of time to support the graft during the remodeling process (e.g. 1-6 weeks after surgery) and may then be configured to undergo partial and/or complete absorption by the body and/or dissolution after external sheath is no longer beneficial (e.g. about 12 weeks after surgery or more). The material and dissolution/absorption times may be selected to avoid chronic inflammation due to foreign body response. Additionally, or alternatively, the external sheaths disclosed herein may comprise and/or be configured to elute one or more specific bioactive drugs and/or medications that may be active to, for example, minimize immune and/or inflammatory responses and/or promote favorable graft remodeling. For example, an external sheath may be configured to elute immunomodulators such as everolimus, paclitaxel, clodronate, or sirolimus to, for example, prevent vasculopathy of bypass conduits associated with the external sheath and/or treat native diseased vasculature. Additionally, or alternatively, a material of an external sheath disclosed herein may be configured and/or selected so that it degrades into byproducts that do not cause substantial inflammation or harm to the patient's body.

In some embodiments, the external sheaths disclosed herein may comprise a biocompatible material that possesses mechanical strength sufficient to withstand manipulation (about 9.8 Newtons of tugging force) with surgical instruments in the operating room and/or during backbench preparation prior to surgery without compromise to its structural integrity. For example, during surgery, the external sheath may be pinched with forceps during sizing and application to the graft. Additionally, vascular clamps may be applied to the sheath to hold it in place or away from anastomotic sites. Additionally, or alternatively, the external sheaths disclosed herein may be configured to maintain their structural integrity when an aspect of the external sheath is modified as may occur when, for example, one or both ends of an external sheath are trimmed, or cut, using a knife or scissors so that the external sheath may be appropriately sized and/or configured to patient anatomy prior to and/or following implantation.

In some embodiments, the external sheaths disclosed herein may be configured to be elastic and withstand stretching while being resistant to tearing. For example, the external sheaths disclosed herein may be configured to stretch (e.g., during an implantation procedure) and then recoil to their original dimensions after manipulation or maintain their new dimensions (following stretching) when the external sheath is, for example, stretched to conform to graft length or diameter. Alternatively, in some embodiments, the external sheaths disclosed herein may be rigid and/or resistant to plastic deformation by vascular clamps or manual pressure applied during surgery.

In some embodiments, the external sheaths disclosed herein may comprise a biodegradable and/or bioresorbable material that is mechanically load-bearing following surgical implantation and later gradually becomes mechanically non-load bearing as the external sheath degrades and/or is reabsorbed over time. In some instances, a desired time period of mechanical load bearing, a degree of gradualness to the stress offloading, and/or a timeframe for degradation and/or resorption of the external sheath may be tailored to specific applications, patient anatomy, arterial characteristics, conduits, and surgical locations by altering the design, geometry, and material composition of sheath assemblies. Often times, the material and degradation byproducts of the bioresorbable and/or biodegradable external sheath disclosed herein may be biocompatible, non-toxic, and/or non-inflammatory.

In some embodiments, the external sheaths disclosed herein may be configured (e.g., degradation rate, an absorption rate, design and/or geometry) to retard, or eliminate, atrophy of grafted tissue and/or promote successful graft “arterialization.” Additionally, or alternatively, the external sheaths disclosed herein may be configured and/or designed to be specific to a tissue type used for a graft, a location and/or type of vessel being grafted, a size of the vessel being grafted, and/or a condition of the vessel from which a graft has been extracted. For example, for saphenous vein grafts, an external sheath may be configured to dissolve and/or be absorbed in as little as 8 days or as long as 2-6 weeks depending on, for example, a degree of mechanical support that may be needed to promote successful graft remodeling and/or “arterialization” and thus reduce risk of graft failure. In one example, an external sheath configured for cooperation with a saphenous vein graft may be configured to maintain mechanical load bearing (i.e., not dissolve/absorb or minimally dissolve/absorb) in the first 1-4 weeks following implantation and gradually dissolve and/or be reabsorbed to become gradually less load-bearing over time, with complete dissolution and/or resorption occurring in a further 2-4 weeks (or as long as 3-4 months) after implantation.

In some embodiments, the external sheaths disclosed herein may be selected and/or designed to accommodate a geometric feature (e.g., size and/or shape) of a graft to which an external sheath is applied and/or mounted so that, for example, an inner diameter of a selected and/or designed external sheath may be the same size as, or slightly larger than, an outer diameter of a blood vessel to which the external sheath is applied. For example, saphenous vein conduits may range from diameters of about 2-6 mm so an external sheath used with a saphenous vein conduit may have an internal diameter ranging from about 2-8 mm. In another example, radial arterial conduits may range from about 3-4 mm so an external sheath used with a radial arterial conduit may have an internal diameter ranging from about 3-6 mm. It is appreciated that various other dimensions may be used according to a given application. Additionally, or alternatively, the external sheaths disclosed herein may be configured to provide uniform, or nearly uniform, radial support along a length of the graft and, in some cases, may extend beyond a length of the graft on one or both sides. A geometric metric of a blood vessel to which the external sheath is to be applied may be derived from, for example, patient-specific medical imaging (e.g., CT, MRI, ultrasound) either before or during surgery. Additionally, or alternatively, in some embodiments, a plurality of external sheaths with varying interior and/or exterior sheath diameters may be provided to a clinician and/or surgeon so that the clinician and/or surgeon may select an appropriately sized external sheath based upon, for example, an image of the patient and/or an intra-operative measurement and/or observation. In other embodiments, the external sheath is made of an elastic material that is either stretched to size or shrunken to fit with natural apposition or slightly oversized (with respect to native environment diameter) to the vascular graft after implantation. In some embodiments, a circular cross section, or a polygonal/conic cross section may be incorporated along a length of an exemplary external sheath. In some instances, a diameter, elasticity, and/or cross-sectional design of an external sheath may be assembled in different combinations depending on the conduit, anatomy, and surgical procedure to which it is applied.

In some embodiments, the external sheaths disclosed herein may be designed and/or configured to adapt to different degrees of curvature depending on, for example, patient anatomy, the surgical site, or surgical procedure being performed. For example, in peripheral vascular bypass surgery a proximal end-to-end anastomosis may require no curvature while a distal end-to-side vascular anastomosis may require slight curvature of an assembly of a graft and an external sheath. In the example of CABO surgery, saphenous vein grafts must take much more significant bends conforming to the curvature of the heart, particularly in the left and right lateral wall anatomic territories. In some embodiments, the elastic properties of the external sheath material may be configured to allow an assembly of a graft and an external sheath to bend or curve without causing kinks to the graft positioned within the external sheath.

In some embodiments, the external sheaths disclosed herein may be configured and/or designed to have a capacity for lengthwise compression and/or expansion so that, for example, an overall length of an external sheath may be shortened and/or expanded, respectively. In some cases, lengthwise compression and expansion of an external sheath may allow for sufficient space to perform vascular anastomoses during surgery while also ensuring that the vascular conduit is supported along its length at the conclusion of the procedure. Once applied, compression to the external sheath may also allow the surgeon to, for example, place repair sutures at sites of anastomotic bleeding and/or re-perform anastomoses as required. In some instances, a larger degree of lengthwise compression may facilitate ease of sheath application, whereas a smaller degree of lengthwise compression (e.g., 1 cm reduction in length) may be sufficient for anastomotic repairs. In some embodiments, lengthwise compression of an external sheath may be achieved and/or maintained via one or more elastic properties a material comprising the external sheath. At times, one or more of the external sheaths disclosed herein may demonstrate elastic recoil back to a set dimension following compression and/or may be pliable and/or maintain an expanded or compressed dimensions until adjusted further.

In some embodiments, the external sheaths disclosed herein may include a hollow tube comprised of a series of elements that form the tube that may be configured to, for example, provide a required amount of radial support while accommodating necessary curvatures and/or deformation of an assembly of the graft and external shaft that may be required for individual patients and/or during surgery. Exemplary elements that may comprise the tube include, but are not limited to, spirals, sequential rings connected along the length of the tube, sequential rings connected along the length of the tube with angled struts, and a lattice structure of tessellating polygons that, in some cases, may be configured to accommodate length compression and/or expansion. Exemplary geometries for external sheaths are provided in the figures and are discussed herein.

In some embodiments, an external sheath may be stored (in an external sheath storage device) and/or provided to a surgeon in a compressed form and, at times, may be expanded and/or further compressed radially or lengthwise, for example, upon removal from a package prior to use and/or throughout the course of surgery. Release of radial compression exerted on some of the external sheaths disclosed herein may cause the external sheath to expand to a full or an oversized diameter for application to, and/or insertion of, a vascular conduit. Additionally, or alternatively, release or restoration of lengthwise compression may be performed, for example, following completion of anastomoses or prior to placing anastomotic repair stitches. In some embodiments, the external sheath storage device may be adjusted by the primary surgeon without need for a surgical assistant, using either full or partial release of sheath compression to achieve desired sizing and position of the graft support. In other embodiments, the sheath storage device is designed to be held by a surgical assistant under partial compression in order to allow for completion or repair of vascular anastomoses after the sheath has already been applied around graft.

The external sheaths disclosed herein may be manufactured using any appropriate method including, for example, 3D printing, stamping, injection molding, and/or combinations thereof. In some embodiments, the external sheath may be manufactured using a single or multiple layers of biocompatible material with a unibody, or multi-part, construction. For unibody construction sheaths, unique combinations of pattern design and material properties are incorporated to achieve surgical maneuverability including radial and lengthwise compression in the absence of mobile joints or multiple layers may include, for example, any of the following or any combination thereof:
Hollow tubular design comprised of sequential circular rings connected along the length of the tube with angled, interconnecting, struts. Interconnecting struts between sequential rings may be oriented at varying offset angles, angular displacements, and thicknesses. In some cases, angled interconnecting, struts positioned between sequential rings of a hollow tubular design may be aligned in the same direction and angle to produce a “spiraling effect” when compressed. Alternatively, angled interconnecting struts positioned between sequential rings oriented in opposing angles and directions between alternating rings, which may, for example, minimize graft torsion during compression.
Latticed design of tessellating polygons extending along the tubular surface. Examples of tessellating pattern designs include diamonds and trapezoids, as well as normal, auxetic, and concave hexagons.
Latticed design with tessellating patterns containing offset curves and waved shapes directed along the length of the tubular sheath.
Segmental designs of sequential hollow cylinders connected by struts along the direction of the tubular length. Connecting struts between sequential cylinders include those of thin and straight design, angled design, and undulating or waved designs.

In other embodiments, the external sheath is manufactured (e.g., 3D printed) in multiple layers and/or with a plurality of segments with, for example, repeating, alternating segments repeating or differing segments and assembled for desired properties of a given procedure. For example, a rigid and stiffer tubular sheath segment may be utilized at a straight proximal end-to-end anastomosis then combined with a more flexible sheath segment to support curvature at a distal end-to-side anastomosis. For multilayer or multi-segment external sheaths, unique combinations of design, material properties, and/or 3D printing techniques may be incorporated to, for example, achieve surgical maneuverability while adequately supporting a graft using, for example, one or more of the following assemblies comprising one or more of:
linked layers, fibers, or segments joined at the point of fabrication, some embodiments with no further assembly required after 3D printing.
interlocking loops, links, and/or rings, of consistent and/or varying geometries arranged longitudinally in a chainmail-like fashion to form an external sheath configured to cover and/or support all, or a portion, of a graft. Individual loops, links, and/or rings of these assemblies may comprise include interlocking loops, links, and/or rings of varying geometries may be arranged within an external sheath in order to, for example, modulate desired stiffness and compressibility along a length of an external sheath.
3D printed fabrics manufactured, or printed, using a varying infill of thick layers and/or printed using an intrinsically flexible material. These 3D printed fabrics may be printed in a tubular form of a finished external sheath and/or assembled (e.g., sewn or glued) together to form a finished external sheath. In some embodiments, the 3D printed fabrics may be sewn into a finished external sheath on an individualized basis so that, for example, an external sheath may be customized to fit a particular graft and/or conduits.
3D printed fibers into braided or woven into external sheaths. These external sheaths may incorporate different fiber alignments to emphasize either radial or lengthwise compression and expansion,
individually printed segments (e.g., unibody, braided, stiff) with similar and/or distinct mechanical properties may be printed at the same time into a single external sheath and/or printed separately and sewn together during, for example, back table preparation in the operating room so that a multi-segment external sheath may be, for example, customized to a particular patient and/or application.

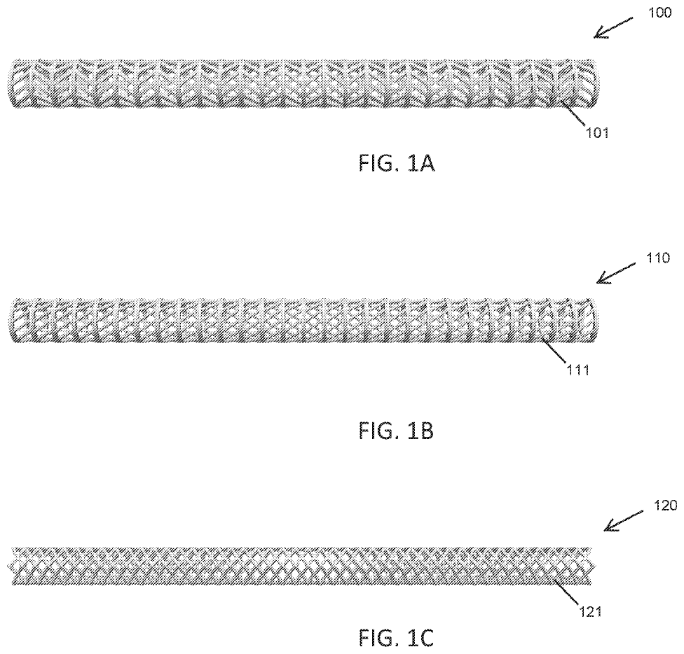

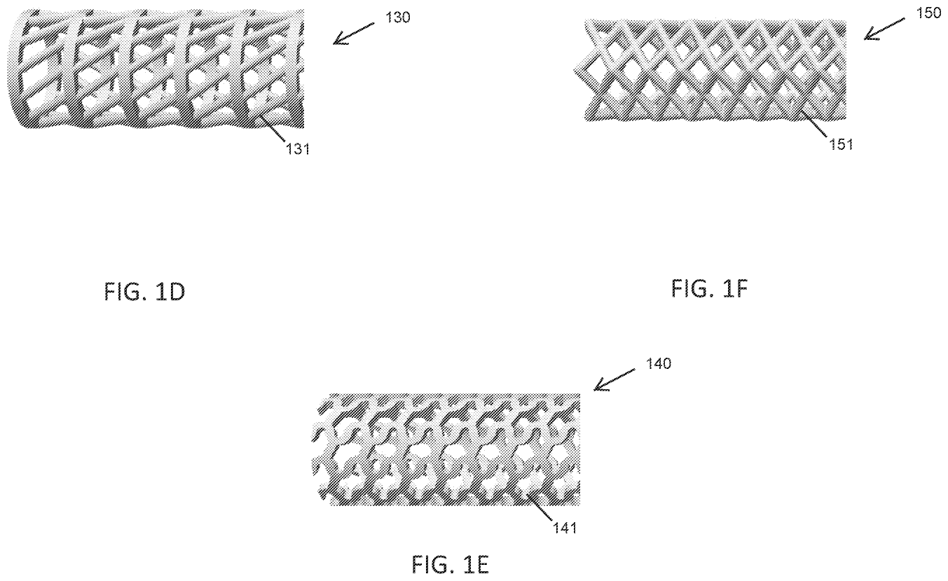

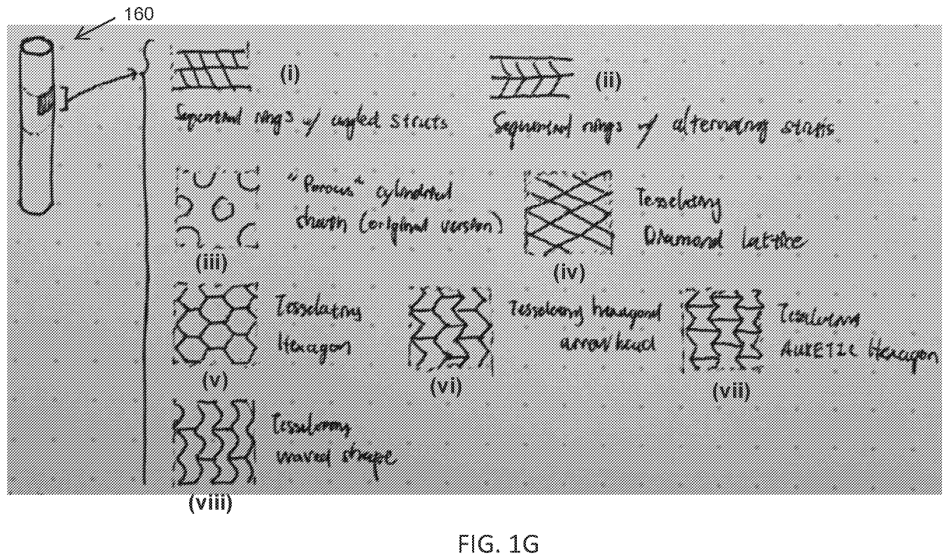

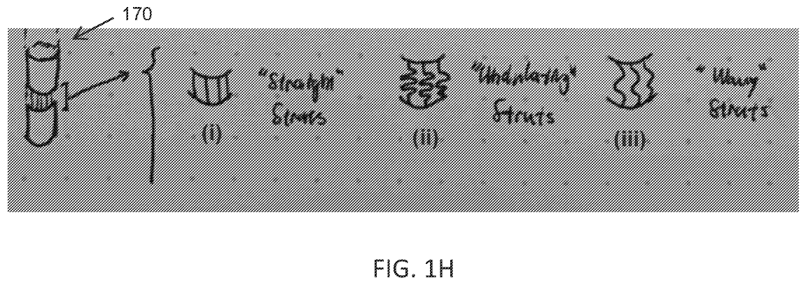

FIGS. 1A-1H provide illustrations and renderings of exemplary external sheaths with various geometrical and/or strut designs as described herein including sheath 100 having alternating struts 101 (FIG. 1A), sheath 110 having non-alternating struts 111 (FIG. 1B), sheath 120 having crisscrossed struts 121 that form a pattern of repeating diamonds (FIG. 1C), sheath 130 having straight struts 131 (FIG. 1D), sheath 140 having smoothly undulating, struts 141 (FIG. 1E), and sheath 150 having sharp-angled undulating struts 151 (FIG. 1F). FIG. 1G provides an exemplary sheath 160 and shows detailed views of a variety of designs and/or arrangements for struts that may be included in an external sheath as disclosed herein. In particular, the designs depicted in FIG. 1G include i) sequential rings with angled struts, ii) sequential rings with alternating struts, iii) a porous material (e.g., holes of any suitable size, 2-100 μm), iv) tessellating diamond lattice, v) tessellating hexagonal lattice, vi) tessellating hexagonal arrowhead, vii) tessellating auxetic hexagonal, and viii) tessellating waved shaped. FIG. 1H provides another exemplary sheath 170 and shows detailed views of strut designs including straight struts, undulating struts, and wavy struts.

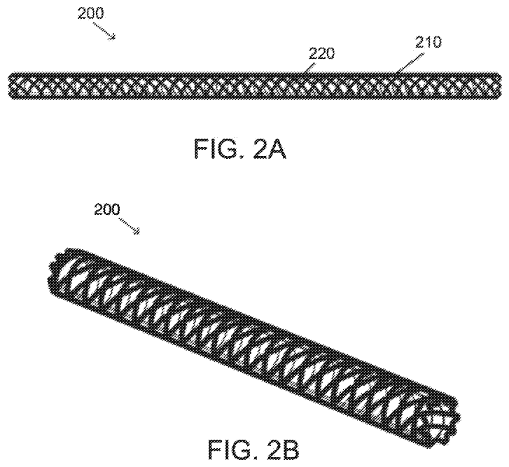

FIG. 2A provides a side view of an exemplary external sheath 200 as disclosed herein, wherein the external sheath comprises a tube formed from a material with a diamond-shaped lattice design and FIG. 2B provides a top-perspective view of exemplary external sheath 200. In this example, external sheath 200 is a single-layer tubular-shaped external sheath 210 formed using a suitable material (e.g., polymer, biodegradable material) and is defined in a lattice work having openings 220 (e.g., pores or interstitial spaces, 2-100 μm) within the lattice design. In some embodiments, this design has sufficient strength so that the external sheath can be deployed during the surgical procedure without a removable internal support tube (not shown), which may allow for greater ease in handling without damage to the external sheath, graft, and/or the native tissue and/or better integration into the clinical workflow.

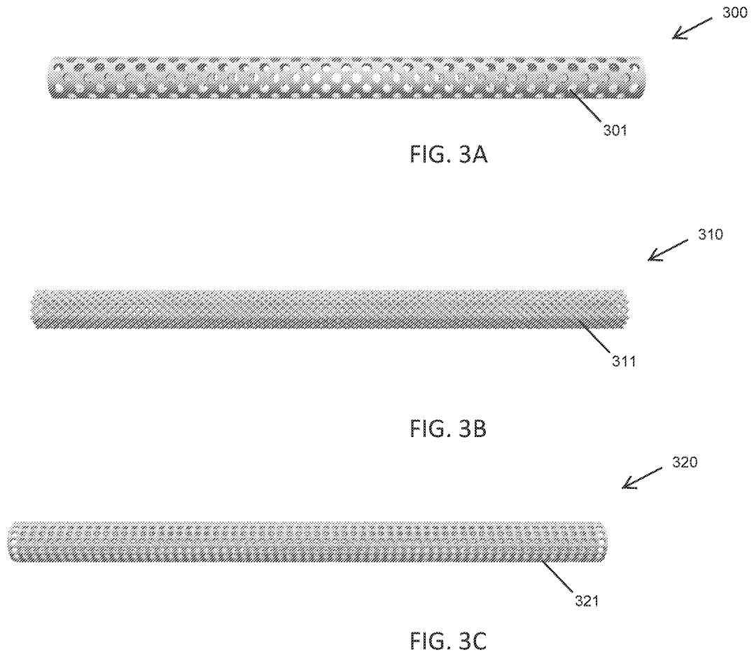

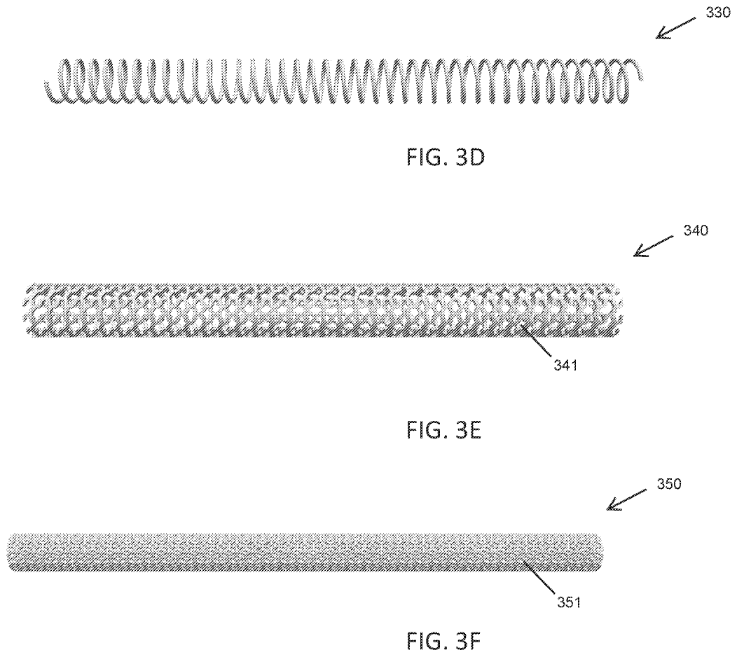

FIGS. 3A-3F provide illustrations and renderings of exemplary external sheaths with different surface patterns including sheath 300 having a pattern of circular holes 301 (FIG. 3A), sheath 310 having a pattern of diamond-shaped holes 311 (FIG. 3B), sheath 320 having a pattern of octagonal-shaped holes 321 (FIG. 3C), sheath 330 formed in a helical pattern (FIG. 3D), sheath 340 formed in a wavy line pattern 341 (FIG. 3E), and sheath 350 formed with a Kelvin Lattice pattern 351 (FIG. 3F).

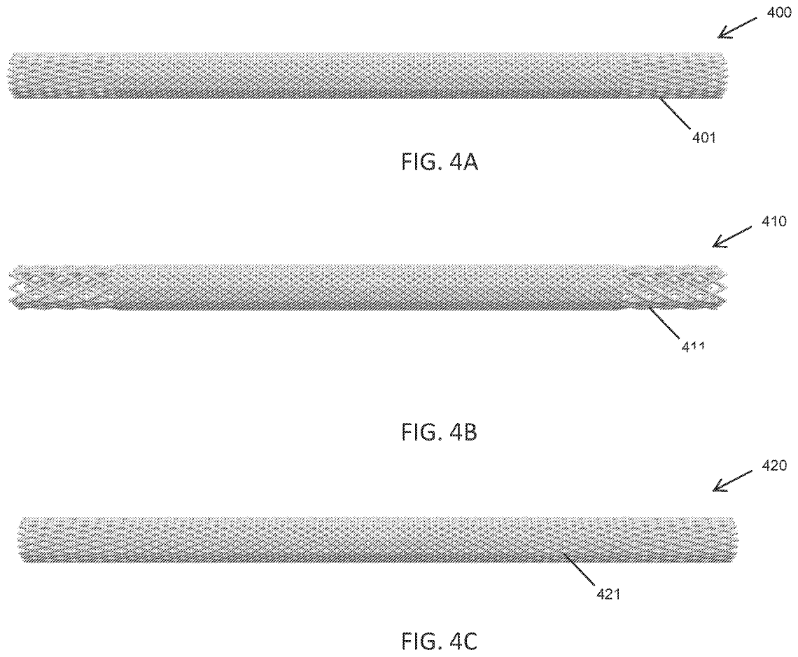

FIGS. 4A-4C provide illustrations and renderings of exemplary multi-segmented external sheaths, wherein a pattern of the material of the external sheath varies along its length. Stated differently, the external sheaths of FIGS. 4A-4C include different segments with a more open/flexible segment on either end of a middle segment that is more densely compact (i.e., holes or openings in a sidewall of the external sheath are smaller) to, for example, provide flexibility at an anastomosis site. In particular, FIG. 4A is a side view of a multi-segmented external sheath 400 with flexible end portions 401 (formed by larger openings), FIG. 4B is a side view of a multi-segmented external sheath 410 with very flexible end portions 411 (formed by even larger sized openings), and FIG. 4C is a side view of a multi-segmented external sheath 420 with a lattice pattern 421 that smoothly transitions from more flexible at either end to less flexible in the middle.

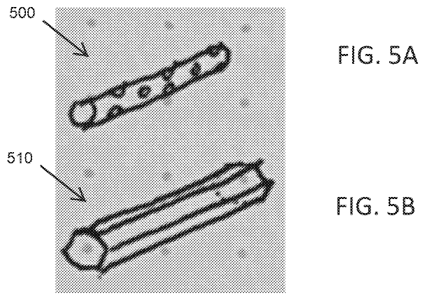

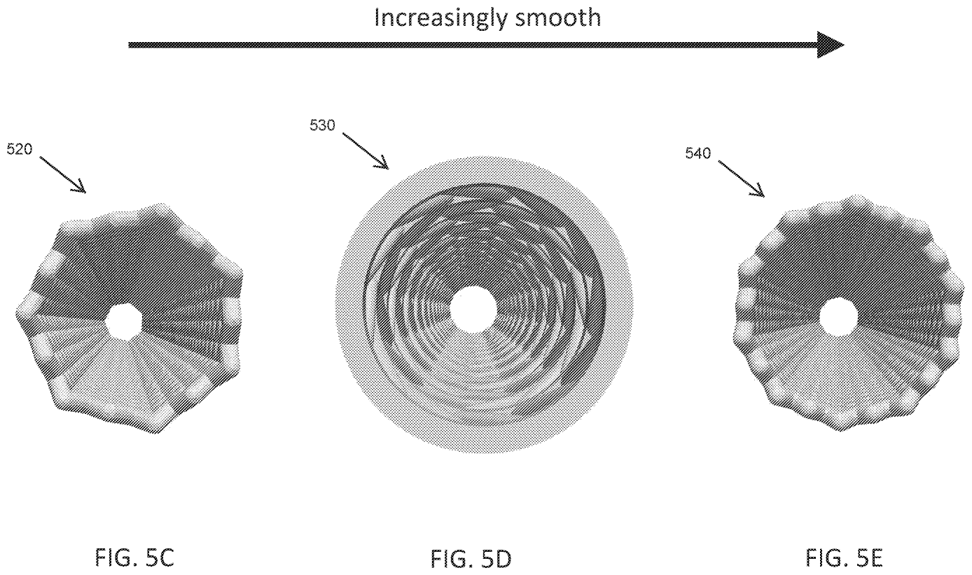

At times, the external sheaths disclosed herein may have a circular, ovoid, hexagonal, decagonal, and/or polygonal vertical (i.e., perpendicular to its length) cross-sectional shape along its length. Some of these exemplary shapes are shown in FIGS. 5A-5E, wherein FIG. 5A provides a side view of an exemplary external sheath 500 with a circular cross-section, FIG. 5B shows a provides a side view of an exemplary external sheath 510 with a hexagonal cross-section, FIG. 5C is a rendering of an end view of an exemplary external sheath 520 with a hexagonal cross-section, FIG. 5D is a rendering of an end view of an exemplary external sheath 530 with a decagonal cross-section, and FIG. 5E is a rendering of an end view of an exemplary external sheath 540 with a circular cross-section.

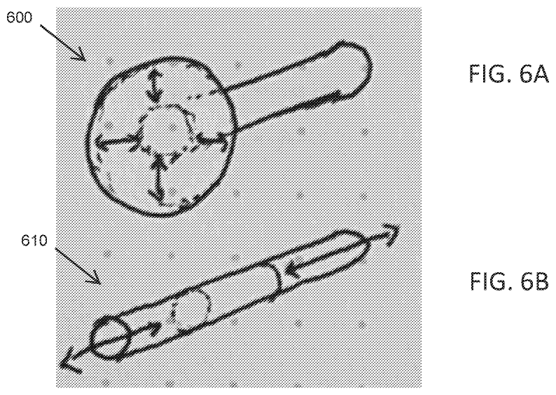

FIGS. 6A and 6B are illustrations that show exemplary external sheaths 600, 610 that are specifically configured for directional compression/expansion, wherein FIG. 6A shows an example of an external sheath design 600 that provides radial compression and expansion and FIG. 6B shows an example of an external sheath 610 that provides longitudinal compression and expansion.

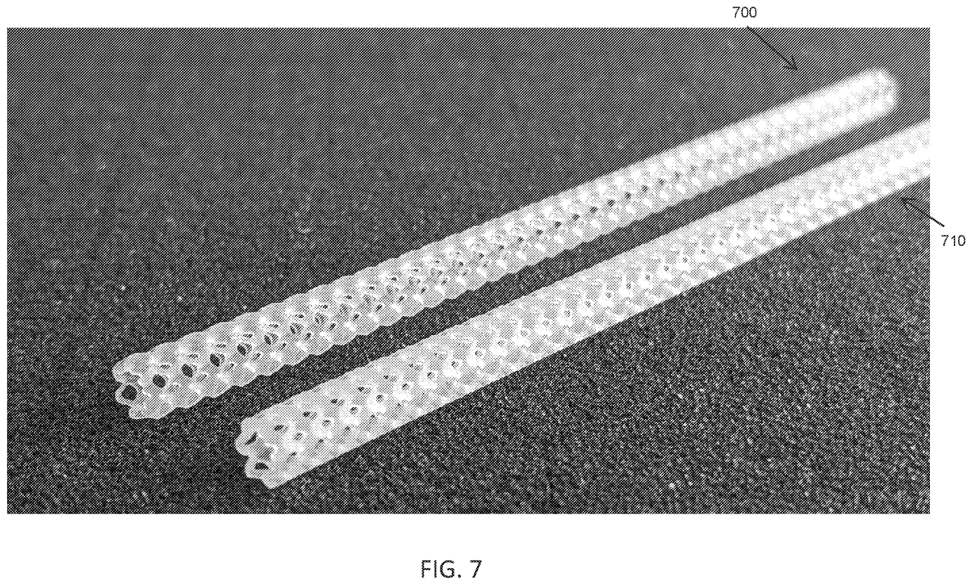

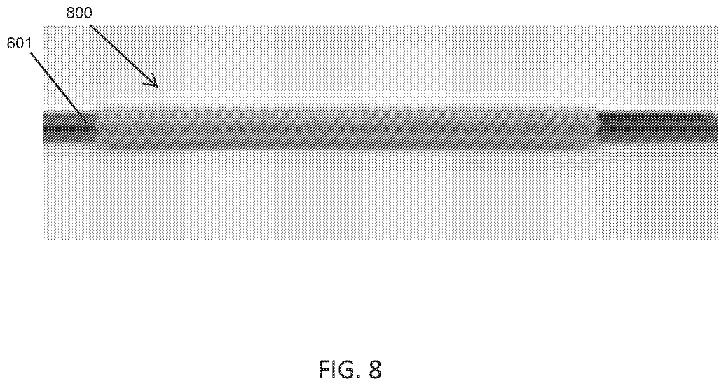

FIG. 7 is a photograph of two exemplary external sheaths 700, 710 as described herein. FIG. 8 is a photograph of an exemplary external sheath 800 with a guide 801 inserted therein along the external sheath's length.

### II. Material Applications

The approaches and aspects described herein represent unique material applications for the construction of external sheaths to prevent graft failure after application in bypass surgery utilizing venous, arterial, and synthetic conduits. The described embodiments incorporate distinct materials and assembly methods, in particular the use and tailoring of poly(propylene fumarate) (PPF) towards patient-specific, anatomy-specific, and surgery-specific utilizations.

In some embodiments, sheath assemblies are based in PPF, a bioresorbable aliphatic polyester capable of being produced in unique architectures to modulate its mechanical behavior. PPF often degrades naturally in the body via hydrolysis and generates nontoxic byproducts (succinic acid, fumaric acid, propylene glycol) that are easily cleared metabolically. In some cases, one or more the mechanical and/or degradation properties of PPF based external sheaths may be easily tuned using, for example, polymer structure and crosslink density that may be available in, for example, PPF material library. In some embodiments, desired in vivo degradation behavior manifests as continued decrease of elastic modulus up to 4-12 weeks after implantation via hydrolytic degradation of ester bonds in the PPF backbone.

In some embodiments, PPF based external sheaths as disclosed herein may be assembled utilizing, for example, thiol-ene chemistry to 3D print external sheaths using PPF with faster build times and more controllable mechanical and degradation tunability. This thiol-ene chemistry-based technique for printing external sheaths using PPF may offer more efficient and customizable sheath assembly as compared to other methods of 3D printing PPF (e.g., with diethyl fumarate (DEF)). In some instances, a technique for 3D printing an external sheath using PPF with thiol-ene based chemistry involves: synthesizing an ABA triblock copolymer elastomer by ring opening copolymerization (ROCOP) from succinic anhydride (SAn), maleic anhydride (MAn) and propylene oxide (PO); adding predetermined amounts of crosslinking agent trimethylolpropane tris(3-mercaptopropionate); dissolving the polymer in ethyl acetate; adding photoinitiator 0.6 wt % BAPO and radical scavenger 0.4 wt % oxybenzone; printing the PPF material into desired geometry via 3D printing; post curing if necessary; and washing excess resin from samples with ethyl acetate followed by isopropyl alcohol. Batch-to-batch variability with this technique is shown to be low and mechanical properties are minimally impacted by sterilization.

At times, resin viscosity and/or construct scaling may be optimized for the 3D printing of PPF into tubular sheaths. For example, 3D printing PPF into self-supporting external sheath structures, ≥70 wt % polymer:ethyl acetate leads to high likelihood of print failure due to the weight of the resin on the 3D printed parts. In some embodiments, 60 wt % and lower polymer resins are shown to reliably improve printing conditions. In some embodiments, external sheaths are additionally scaled between 1.2× to >1.5× in order to compensate for isotropic shrinkage of the 3D printed parts.

In some embodiments, PPF based external sheaths may be printed and/or assembled with varying crosslinking density in order to, for example, match native tissue or exhibit specifically desired mechanical behaviors. For example, alkene:thiol ratios within printed PPF influence crosslinking density and may range from 5:1 up to 100:1 for graft applications. In another example, the alkene:thiol ratio of 20:1 may possess more crosslinking than the 30:1 crosslinking ratio, thus degrading slower and maintaining more mechanical stability with time. In some embodiments, 5:20:5 ABA triblock copolymer may be synthesized and printed with higher alkene thiol ratios (e.g., 40:1, 50:1) to allow for more degradation and softer materials. Higher crosslinking PPF external sheaths may exhibit high elasticity and may, therefore, be highly compressible and recover quickly from mechanical deformation. It is understood that PPF crosslinking density can be used to adjust the mechanical integrity and/or stability and degradation behavior of PPF sheath assemblies to different desired times of external sheath and unloading for the given surgery and conduit to which they are applied.

In some cases, PPF based external sheaths may exhibit tunable degradation behavior through modifications in both crosslinking density and patterned design. For example, latticed designs with more exposed surface area may enable additional infiltration of water into the PPF based external sheath and thus facilitate faster hydrolysis, which may lead to full degradation in as early as 12 days.

In some embodiments, PPF based external sheaths may be assembled in varying combinations of diameter, length, thickness and patterns tailored towards distinct graft and/or conduit sizes and/or desired mechanical properties. In some cases, PPF external sheaths may be mechanically flexible and may be configured so that they do not soften noticeably at physiological temperatures. Additionally, or alternatively, one or more of the PPF external sheaths disclosed herein may not change in compression, shear, and/or tension behavior at physiological temperature in aqueous conditions compared to room temperature conditions in air. Differently sized external sheaths may be manufactured (e.g., 3D printed) with low and/or high cross-linking ratios with the intended purpose being applied to different sized vessels. In some cases, smaller diameter external sheaths may experience larger forces at higher compression, and the difference in mechanical strength between 37° C. and room temperature is more pronounced between the smaller, stiffer samples.

### III. Methods of Use

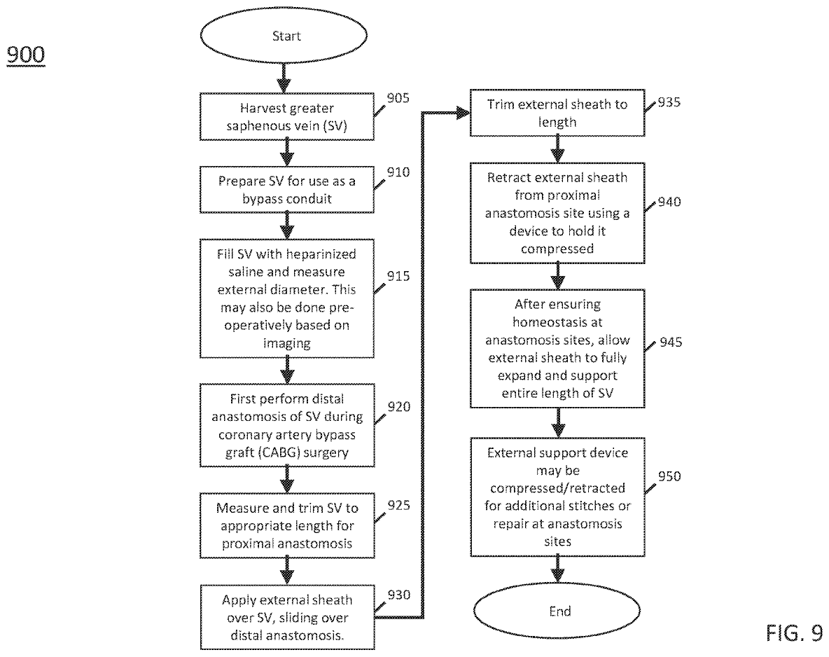

FIG. 9 provides a flowchart of an exemplary process 900 for performing a CABG procedure using an external sheath as shown and described herein. Initially, in step 905, a segment of a patient's greater saphenous vein (SV) may be harvested and prepared for use as a graft and/or cardiac bypass conduit (step 910). Then, the harvested SV segment (also referred to herein as a “graft”) may be filed with heparinized saline and an external diameter of the harvested SV segment may be measured (step 915). Additionally, or alternatively, an external diameter of the to-be harvested SV segment may be imaged (e.g., ultrasound and/or MRI) prior to harvesting to determine an external diameter of the SV segment.

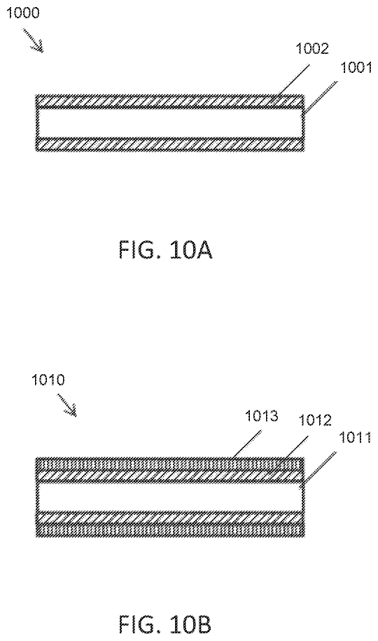

Next, a distal anastomosis of the harvested of SV segment may be performed during the CABG surgery (step 920). Then, the harvested SV segment may be measured and trimmed to an appropriate length for proximal anastomosis (step 925). An external sheath, such as the external sheaths disclosed herein, may then be applied to the harvested of SV segment by, for example, sliding the external sheath over the harvested of SV segment so that the harvested of SV segment is positioned within a central lumen of the external sheath (step 930). In some cases, step 930 may be performed by sliding the external sheath over the distal anastomosis. A result of execution of step 930 may the generation of a graft/external sheath assembly such as a single layer graft/external sheath assembly 1000 shown in FIG. 10A, which is a cross-section view of first single layer graft/external sheath 1000 that includes a graft 1001 and an external sheath 1002, which may be one or more of the external sheaths disclosed herein and/or designed and/or constructed using one or more of the features and/or processes described herein. In some cases, external sheath 1002 of graft/external sheath 1000 may be designed and/or selected to, for example, be compatible with patient anatomy, surgical requirements, promote advantageous graft remodeling, and/or match design specifications that prevent maladaptation and/or improper graft remodeling. In some cases, external sheath 1002 may be formed of materials selected or customized, individually or in combination, to match the properties of the natural vasculature and/or to match design specifications. In some embodiments, external sheath 1002 may comprise biodegradable material so as to provide further reinforcement immediately after surgery and for a short time thereafter (e.g., about 1-6 weeks after or more), and later dissolve/degrade/resorb when additional reinforcement is no longer needed, thereby providing properties for favorable long-term adaptation. In some embodiments, this short period of time thereafter may be 1-6 weeks after surgery. In other embodiments, external sheath 1002 may be configured to biodegrade 3-4 months post-operatively.

Another exemplary result of execution of step 930 is shown in FIG. 10B, which is a cross-section view of a multi-layer graft/external sheath 1010 comprising graft 1011 positioned within a lumen of a first layer 1012. Multi-layer external sheath also includes a second layer 1013 positioned on top of, and/or surrounding, first layer 1012. First and second layers 1012 and 1013 may comprise the same material or different materials having the same and/or different geometries. In some embodiments, the first and second layers provide differing functions of, for example, increased stiffness and drug elution. For example, in some embodiments, second layer 1013 may be configured to provide reinforcement against distension and first layer 1012 may be configured to provide drug elution. In some embodiments, first and/or second layers 1012 and 1013 may be biodegradable (e.g., with different degradation rates), while others are not biodegradable, to provide variability in properties. In some embodiments, multiple layers having differing rates of biodegradability are used to provide design properties that change or adapt over time, thereby mitigating maladaptation and failure after surgery. It is appreciated that the layers and their associated functions could be arranged in any number of ways. Further, it is appreciated that the multi-layer graft support assembly is not limited to two layers and can encompass any number of layers desired.

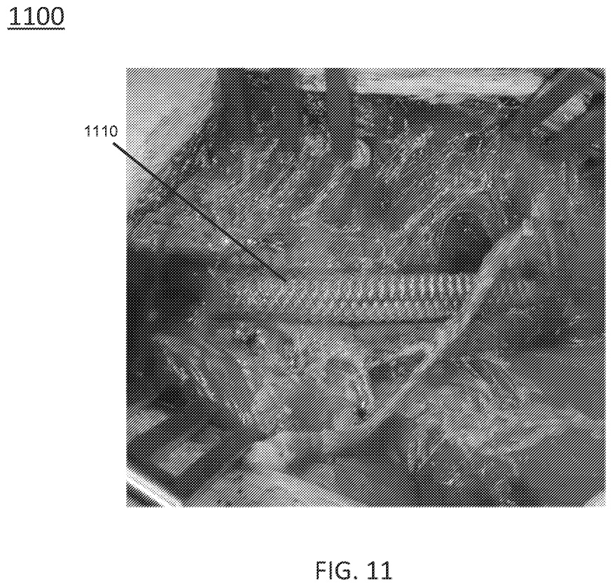

In step 935, the external sheath may be trimmed to a desired length, and, in step 940, the external sheath may be retracted from a proximal anastomosis site using, for example, a device to hold the external sheath compressed. Once hemostasis of the patient is established/ensured, compression on the external sheath may be released thereby allowing the external sheath to fully expand and support the entire length of the graft (step 945). Then the external sheath may be compressed and/or retracted to access surgical sits that may require additional stitched and/or repair to anastomosis sites (step 950). Following step 950, process may end and procedures to complete the CABG may be executed and/or performed on the patient. FIG. 11 provides a photograph 1100 of an exemplary external sheath 1110 positioned over a graft in a manner consistent with execution of process 900.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. As used herein, the term “about” is +/−10%, or +/−20% of the recited value. It is recognized that the terms “comprising,” “including,” and “having,” as used herein, are specifically intended to be read as open-ended terms of art. Any patent publications or references noted is incorporated herein by reference in their entirety for all purposes.

What is claimed is:

1. A vascular sheath or graft assembly comprising:
a tubular external sheath formed of a biocompatible material, the tubular sheath configured with sufficient flexibility to accommodate a vasculature of the patient and sufficient strength along the tubular sheath to maintain blood passage through the graft,
wherein the tubular sheath comprises one or more layers, wherein at least one of the one or more layers comprise a biodegradable or bioresorbable and 3D printed design;
wherein the design comprises a plurality of struts arranged in a pattern to facilitate radial and/or longitudinal compressibility and expandability and surgical manipulation;
wherein the tubular sheath has dimensions and characteristics that are selected or designed to correspond to a specific vasculature of a patient to which the graft is mounted so as to mechanically reinforce the graft for at least a period of time after the surgical procedure so as to mitigate maladaptation and graft failure after surgery.

2. The vascular sheath or graft assembly of claim 1, wherein the 3D printed layer uses thiol-ene chemistry to achieve faster build times and customization of mechanical integrity and stability periods (1-6 weeks) and desired degradation behavior (0.5-6 months).

3. The vascular sheath or graft assembly of claim 2, wherein the 3D printed layer comprises poly(propylene fumarate) (PPF).

4. The vascular sheath or graft assembly of any preceding claim, wherein the design comprises sequential rings connected along a length of the sheath by the plurality of struts.

5. The vascular sheath or graft assembly of any preceding claim, wherein the plurality of struts is arranged to enhance longitudinal compressibility and expandability of the tubular sheath.

6. The vascular sheath or graft assembly of any preceding claim, wherein the plurality of struts is arranged to enhance radial compressibility and expandability of the tubular sheath.

7. The vascular sheath or graft assembly of any preceding claim, wherein the plurality of struts is angled in a same direction or alternating directions.

8. The vascular sheath or graft assembly of any preceding claim, wherein the plurality of struts is arranged in a pattern of tessellating polygons.

9. The vascular sheath or graft assembly of any preceding claim, wherein the plurality of struts is arranged in a pattern comprising any of:
a tessellating diamond lattice,
a tessellating hexagonal pattern,
a tessellating hexagonal arrowhead pattern,
a tessellating auxetic hexagonal pattern, and
a tessellating waved shape pattern.

10. The vascular sheath or graft assembly of any preceding claim, manufactured by chainmail assembly with individual linking parts.

11. The vascular sheath or graft assembly of any preceding claim, wherein the plurality of struts enables a release of immunomodulatory and immunosuppressive drugs with a desired kinetics profile.

12. A method of forming a vascular sheath comprising:
3D printing, using thiol-ene chemistry, one or more layers of a tubular sheath for a vascular graft comprising a tubular external sheath formed of a biocompatible material, the tubular sheath configured with sufficient flexibility to accommodate a vasculature of the patient and sufficient strength along the tubular sheath to maintain blood passage through the graft.

13. The method of claim 12, wherein the 3D printed layer uses thiol-ene chemistry to achieve faster build times and customization of mechanical integrity and stability periods and desired degradation behavior.

14. The method of claim 13, wherein the stability periods is within a range of 1-6 weeks and the desired degradation behavior period is within a range of 0.5-6 months.

15. The method of any of claims 12-14, wherein the 3D printed layer comprises poly(propylene fumarate) (PPF).

16. The method of any of claims 12-15, wherein the design comprises sequential rings connected along a length of the sheath by a plurality of struts.

17. The method of claim 16, wherein the plurality of struts is arranged to enhance longitudinal compressibility and expandability of the tubular sheath.

18. The method of claim 16, wherein the plurality of struts is arranged to enhance radial compressibility and expandability of the tubular sheath.

19. The method of claim 16, wherein the plurality of struts is angled in a same direction or alternating directions.

20. A method of forming an assembly or a jig to enable assembly and mounting of the vascular graft comprising a tubular external sheath formed of a biocompatible material, the tubular sheath configured with sufficient flexibility to accommodate a vasculature of the patient and sufficient strength along the tubular sheath to maintain blood passage through the graft.
